(19) United States
(12) Patent Application Publication
(10) Pub. No.: US 2008/0124716 A1
(43) Pub. Date: May 29, 2008

(54) Method and device for time-effective biomolecule detection

(75) Inventors: Christopher G. Cooney, Severn, MD (US); Matthew J. Lesho, Ellicott City, MD (US)

Correspondence Address: ROTHWELL, FIGG, ERNST & MANBECK, P.C., 1425 K STREET, N.W., SUITE 800, WASHINGTON, DC, 20005, US

(73) Assignee: Northrop Grumman Systems Corporation, Los Angeles, CA (US)

(21) Appl. No.: 11/605,353

(22) Filed: Nov. 29, 2006

(51) Int. Cl. C12Q 1/68; C12M 1/00

(52) U.S. Cl. 435/6; 435/287.2

(57) ABSTRACT

Rapid detection of biomolecules in samples involving biochemical amplification of the target biomolecule is achieved by collecting or separating aliquots of the amplification reaction mixture prior to completion of amplification and assaying these samples as they are collected.

### BACKGROUND OF THE INVENTION

1. Technical Field

The present invention relates to the field of biochemical assay and sample preparation methods thereof. Specifically, the invention relates to a method of rapidly assaying samples by biochemical amplification and subsequent detection. The method includes collecting sample aliquots of an appropriate size for miniature devices such as mass spectrometers, capillary electrophoresis devices, microarrays and the like during or before biochemical amplification and subjecting these samples to assay.

2. Description of the Background Art

Biochemical amplification methods such as polymerase chain reaction and the like are known in the art and are extremely useful to enable detection of small amounts of a biological molecule such as a nucleic acid in a sample. These methods allow molecules present in a sample to be amplified so that they are present in sufficient quantity to be detected in the sample using conventional detection methods.

Since the amount of the material present in the original sample or in the amplified sample usually is not known prior to its assay, the optimal time of the amplification reaction cannot be known in advance. Therefore, it is necessary to amplify all samples using a reaction time long enough to ensure that those samples containing the target molecule at the lowest limit of detection are amplified enough to detect and/or identify the target. The result is that in samples in which there is a low concentration of target, the target is detected. An unfortunate disadvantage, however, is that samples that contain a high concentration of the target also are amplified for the maximum time because these samples are not known in advance. Thus there is an unavoidable and undesirable delay in detecting “hot” samples in which the target material is present in large amounts.

There is a need in the art for methods that avoid such delays and are able to detect “hot” samples which do not require lengthy amplification times rapidly and without unnecessary over-amplification, while still allowing less concentrated samples also to be detected with longer amplification times.

### SUMMARY OF THE INVENTION

Accordingly, embodiments of the present invention provide a rapid assay method for detection of a target biomolecule, which comprises (a) providing a sample for analysis; (b) optionally subjecting said sample to analysis for detection of said target biomolecule; (c) subjecting said sample to biochemical amplification by combining said sample with biochemical amplification reagents to form a biochemical amplification reaction mixture and subjecting said biochemical amplification reaction mixture to conditions wherein biochemical amplification can take place; (d) simultaneously with (c) collecting at least one sample aliquot of said sample during biochemical amplification; and (e) subjecting said sample aliquot to analysis for detection of said target biomolecule. In preferred methods, the target is a nucleic acid and amplification is performed using the polymerase chain reaction or isothermal nucleic acid amplification methods such as strand displacement amplification, the exponential amplification reaction (EXPAR) and abscription. Preferred detection methods include capillary electro-phoresis mass spectrometry.

Preferably, sample aliquot collection comprises subjecting said biochemical amplification reaction mixture to fluid transport along a fluid conduit and separating discrete volumes of said biochemical amplification reaction mixture from each other to form aliquots by introducing an immiscible fluid at intervals in said fluid conduit. Sample aliquot collection may occur prior to said biochemical amplification, after said biochemical amplification begins (during or after amplification), or both.

In other embodiments, the invention provides an assay device for amplification and rapid assay of a sample for presence of a biomolecule target which comprises (a) a hollow fluid conduit comprising a first open end, a second open end and an opening in said conduit between said first end and said second end to admit a fluid into said fluid conduit; (b) a means for producing fluid flow in said fluid conduit in the direction from said first end to said second end; (c) a means for introducing an amplification reagent mixture into the first end of said fluid conduit and a means for introducing said sample into the first end of said fluid conduit to mix said amplification reagent mixture and said sample together to form a reaction mixture in said fluid conduit; (d) a reaction chamber disposed in said fluid conduit between said first end and said second end, wherein said reaction chamber provides conditions under which amplification of said biomolecule target can occur; (e) an aliquot collection means that introduces a fluid into said fluid conduit at intervals, wherein said fluid is immiscible with said reaction mixture and wherein said fluid separates said reaction mixture into discrete aliquots of reaction mixture; and (f) a detector, detachably connected in a fluid conducting manner to the second end of said fluid conduit. Preferred aliquot collection means are selected from the group consisting of a fluid injector, an electrostatic droplet splitter and an electrolytic gas generator and preferred detectors are selected from the group consisting of a mass spectrometer, a capillary electrophoresis device with an optical detector and a microarray.

### BRIEF DESCRIPTION OF FIGURES

FIG. 1 is a schematic diagram of an embodiment of an assay device according to the present invention.

### DETAILED DESCRIPTION OF THE INVENTION

Assay of biological samples for specific nucleic acids often involves a signal generation method (a biochemical, chemical or specific binding reaction that results in or allows a detectable signal). Amplification of the target molecule in the sample prior to its detection or binding of a target DNA molecule to a microarray for detection using a dye (for example an intercalating dye that identifies double-stranded target-probe duplexes) are examples of known techniques for target signal generation. In the vast majority of cases, the amount or concentration of the target in the sample (if any) is not known prior to assay. Common practice, therefore, for assay of an unknown amount of target in a biological sample to be amplified, is to assume that all samples contain the smallest detectable amount of target and therefore to amplify all samples for the time required to allow sufficient amplification of this amount of target. This ensures that every sample that contains the target molecule in amounts above the lowest limit of detection will be detected, but has the disadvantage that all samples must undergo the longest possible amplification prior to assay. Amplification reactions typically represent the rate-limiting step in sample analysis, and require anywhere from tens of minutes to hours to complete.

Embodiments of the present invention provide methods which allow the detection of target in a sample as amplification proceeds, without waiting for completion of a lengthy and potentially unnecessary amplification reaction. These methods allow a positive result to be obtained more quickly for any sample that contains target in a concentration above the lower limit of detection of the system with which it is integrated. In general, the methods involve sampling and assay of the reaction mixture as amplification proceeds.

“Target” or “target molecule,” as used herein, refers to a molecule which is to be detected in a sample using an assay or detection system. A target therefore can be any detectable molecule. A particular target may or may not be present in any particular sample, and may be present in differing amounts in different samples. For preferred embodiments of the present invention, target molecules include nucleic acids such as DNA, cDNA, and RNA, proteins and peptides, toxins and PNAs.

“Amplification,” as the term is used herein, includes any signal generation method which is or may be used to increase the amount or concentration of the target molecule in a sample prior to detection of the target.

“Signal generation” is a biochemical, chemical or binding reaction that results in or allows a detectable signal to be created, which may or may not involve amplification. Examples of amplification methods include, but are not limited to, methods for amplification of nucleic acids such as polymerase chain reaction, ligase chain reaction, strand displacement amplification, helicase dependent amplification, rolling circle amplification, loop mediated isothermal amplification and the like. For production of a fluorogeric or chromogeric substrate, methods involving alkaline phosphatase or horseradish peroxidase are suitable and known in the art.

“Detection,” as the term is used herein, refers to any method of detection of a target molecule known in the art and may include, but is not limited to, mass spectroscopy, capillary electrophoresis, electrophoresis, microarray (biochip) detection, immunochemical methods, real-time fluorescence, amperometry, voltammetry and cyclic voltammetry.

Samples containing more highly concentrated target can be identified without delay by removing and collecting small aliquots from the overall reaction volume while the reaction continues to amplify. An “aliquot” or a “sample aliquot” is a small representative fraction of a sample which may be assayed in lieu of using the entire sample. These sample aliquots are assayed upon collection and before and/or after the signal generation reaction is complete. If the original sample contained sufficient target such that the concentration in the aliquot is detectable after no amplification or only partial amplification, a positive result can be obtained far sooner than could be achieved otherwise. According to embodiments of the invention, therefore, sample aliquots are removed from the amplification reaction mixture and carried to a detector as the reaction proceeds so that if the detector identifies the sample as positive, amplification need not continue. Alternatively, the amplification reaction mixture containing the sample is divided into multiple aliquots which are subjected to amplification conditions individually for differing times and then assayed for the target. If the original sample contains a high number of copies of the target initially, the sample can be identified as positive more quickly. If the original sample contained a low concentration of the target initially, the target may not be detected in a sample aliquot subjected to limited amplification. In these cases, amplification would be allowed to proceed further and the reaction mixture re-assayed by collection of another aliquot.

Rapid detection and identification of “hot” samples, which contain large amounts of target, is especially useful in detection of biohazards, for example E. coli in foods or any bioagent with potential for use as a bioweapon, such as anthrax, smallpox and the like. Rapid identification of “hot” samples in these applications provides important information without delay so that appropriate action can be taken. The methods of the invention therefore are particularly advantageous in applications for monitoring and for detection of nucleic acids, such as those designed to identify a biohazardous organism, but may be used in any application where rapid identification of a target biomolecule is desirable.

Amplification reactions produce target amplicons in a time-dependent fashion. This rate is based on the concentration of the species being amplified rather than on the total amount or copy number present in the sample volume except in cases of amplicon saturation or limitations in mass transfer kinetics. This characteristic allows one to remove volumes of fluid for testing in as the reaction progresses without negatively affecting the reaction rate. Biochemical amplification of a sample usually takes place in a volume ranging from about 25 microliters up to hundreds of microliters. Many detection methods require only a sample having sufficient material for the detection limit of the device with sample volume a secondary consideration. The volumes of samples for detection vary, but often are a great deal lower than the amplification reaction volume, and may be as low as about 10 nanoliters for some miniaturized detectors.

The volume of sample aliquots removed from the amplification reaction in the inventive methods will depend on the volume required for detection by the detection method or device being used and on the volume available in the amplification reaction vessel. It is contemplated that the invention can accommodate any convenient sample aliquot volume amount, however preferred volumes for collection from the amplification reaction mixture range from about 5 pl to about 100 μl and most preferably from about 50 pl to about 10 μl or about 500 pl to about 1 μl per sample aliquot. Most preferred volumes are sufficient for rapid, miniature detection devices such as mass spectrometers or capillary electrophoresis devices and range from about 100 pl to about 200 nl, so that the amount of initial sample required for amplification and analysis is small and the reaction can take place in the smallest convenient volume. Assuming that the reaction vessel is well mixed, removal of one or more sample aliquots totaling up to about 10% of the reaction volume or less, or preferably about 5-9% of the reaction volume, would not compromise the lower limit of detection of target in the initial sample because the concentration of the target in the reaction mixture remains the same despite the volume change and at least 90% of the sample would still be available after aliquot removal for an endpoint reading. Such an assay of the remaining sample can be performed if none of the sampled aliquots resulted in a positive reading or as a quality control even if one or more aliquots contained detected target. Thus, methods which involve removing a total of 10% of the reaction volume or less are preferred. Therefore sample aliquots can total in this range for some embodiments of the invention, but can be higher, including 20% or even up to nearly 100% of the total reaction volume.

One sample aliquot or more than one can be removed from the reaction vessel during amplification of the target in a sample according to different embodiments of the invention. For example, in applications where it is desirable to immediately detect extremely concentrated samples of a molecule, such as in biohazard monitoring applications, one sample aliquot collected before or near the start of the amplification reaction may be sufficient to rapidly detect a “hot” sample. For many applications, removal of one sample aliquot (“episodic removal”) is sufficient to accomplish the goal of rapid identification of a concentrated sample. In additional embodiments of the invention, sample aliquots preferably are removed according to a predetermined periodic schedule (“periodic removal”), for example about every 30 seconds to every 5 minutes or preferably about every 1 minute to every 4 minutes and most preferably about every minute.

Removal of sample aliquots for assay in amplification embodiments of the invention can begin as soon as the amplification reaction begins and continue throughout the course of amplification until the reaction is complete. In preferred embodiments, aliquot collection begins about 30 seconds after amplification begins. Preferred schedules for aliquot removal begin at 30 seconds and continue until about 10-30 minutes have elapsed, for example about 20 minutes. Sample aliquots can be removed at any interval which is convenient, for example every 30 seconds, every 10 seconds, every 15 seconds, every minute or every 2-5 minutes.

Additional embodiments of the invention provide an initial pre-screening method wherein a sample aliquot is collected and assayed prior to amplification. These methods allow very rapid identification of samples which are sufficiently concentrated for detection of target without amplification. This pre-screening aliquot collection is the sole sample aliquot removal from a sample or alternatively is accompanied by removal of one or more further sample aliquots during amplification. If the sample contains sufficient target for detection in the absence of any amplification, the signal generating reaction can serve as a check to confirm that the sample is indeed positive.

According to a further embodiment, multiple sample aliquots are removed from a sample during amplification and assayed as part of a quality control check which confirms positive results and identifies false positives. Therefore, when a first or subsequent sample aliquot is identified as containing the target, further amplification and testing may be halted, or may continue. In the case of a false positive, subsequent sample aliquots taken from the sample will be negative or fail to increase with increased amplification time, whereas in the case of a true positive, subsequent sample aliquots will continue to be positive, and where the detection means is quantitative, will show higher and higher amounts of the target in the sample aliquots as amplification proceeds.

When sample aliquots are collected over time and subjected to analysis, the area under the concentration curve of the detector should be proportionate to the amplification rate. Therefore, the invention can provide a quality control mechanism to confirm positive results with one or more subsequent sample aliquot prior to reporting a positive result, and also can be used to determine amplification rate of the reaction as it takes place, using a sample or using a control solution with known target concentration.

An additional embodiment of the invention relates to a method wherein sampling of the amplification reaction mixture is continuous. Real-time or near real-time detection can be achieved by continuously flowing the reaction mixture from the amplification vessel to a detector or to a device for delivery to a detector. For example, reaction mixture can be delivered to an electrospray chamber which continuously ejects particles into the analyzer of a mass spectrometer or a capillary electrophoresis channel. Such a configuration with continuous sampling provides continuous feed-back information until a peak in the mass spectrum or electropherogram (or other detection method) is positively identified. Continuous sampling methods include those in which the amplification reaction mixture is sequentially divided into multiple small volumes of reaction mixture which serve as sample aliquots, where each sample aliquot is individually subjected to amplification conditions to allow amplification of each sample for a different time, for example a longer time for each successive sample aliquot, and then subjected to a detection step.

Signal generation methods are known in the art to those of skill. Any of these methods are contemplated for use with the invention. Typically, a combination of biochemicals is incubated at a constant temperature or subjected to temperature cycling to induce biochemical interactions and synthesis that result in the generation of additional copies of the original target or marker molecules when amplification is desirable prior to detection by generation of a signal. Signal generation methods can involve reaction buffers, enzymes, fluorescent or non-fluorescent markers and recognition molecules such as primers and probes. Preferred signal generation methods are those which amplify the target molecule to an extent which enables its detection and occur in a fluid medium that can be subdivided for collection of sample aliquots. The reaction mixtures most preferably are well-mixed during amplification so that any sample aliquot collected from the body of the reaction mixture will accurately represent the target concentration in the sample and the concentration of target in the sample will not be changed by removal of a sample aliquot.

Amplification methods which can be used to the best advantage with the invention amplify a short segment of a nucleic acid which is unique to a biological organism or a class of biological organisms to be detected. For example, pre-determined genomic regions (DNA sequences) of the plasmids of Bacillus anthracis may be amplified in some embodiments of the invention. Multiplex amplification methods, in which more than one target molecule is amplified simultaneously, also are contemplated for use with the invention, in combination with detection methods capable of specifically detecting the multiple amplicons, either individually or as a class. One suitable amplification scheme involves use of a polymerase and reaction buffers that include magnesium. The reaction is isothermal and proceeds at 55° C. When using this method, reagent removal has minimal thermal impact on the reaction kinetics.

Detection methods likewise are known in the art. Any detection method capable of identifying a biomolecule, generically or specifically, may be used with embodiments of the invention. For example, a detection method which detects DNA in a sample may be used, or a detection method which specifically detects a unique DNA molecule having an unique sequence may be used. Detection methods for use with the invention advantageously provide a rapid result and require small sample sizes for accurate detection.

Alternatively, embodiments of the invention can employ a detection method in which samples from the reaction mixture are injected serially into a detection chamber such as for capillary electrophoresis. Capillary electrophoresis traditionally is performed in channels that are 20 microns by 50 microns in cross-section and 2 to 8 cm in length, however, any suitable device with convenient dimensions may be used. Free solution capillary electrophoresis has the ability to discriminate short oligonucleotide molecules such as are produced in strand displacement amplification assays. The small oligonucleotide products of SDA can be separated in less than 3 minutes or less than 2 minutes, because the reaction products can be as short as dimers and trimers.

Detection methods which require only a very small sample size for operation are preferred. Therefore, preferred highly miniaturized detectors are those such as miniaturized mass spectrometers that typically require sample volumes below 10 μl, capillary electrophoresis detectors that typically require sample volumes of about 100 pl, and accelerated microarray detectors that require sample volumes of about 250 μl are preferred. The most preferred detection method is a capillary electrophoresis device that requires a sample volume as low as 100 pl to about 10 nl.

Mass spectrometric methods can discriminate among many types of organisms. For example, these methods can specifically distinguish between individual lethal bioagents and innocuous organisms which also may be present in a sample for testing. In mass spectrometry, this discrimination is based on precise resolution of amplified products using signal generation molecules coupled to a molecule such as an antibody that binds to or interacts with toxins. The signal generator molecules are designed to be detected by mass spectometry. Additionally, when using mass spectometric detection methods, a small initial aliquot of the sample can be used for direct toxin detection by fragmenting the peptide target into amino acids and computing the mass of the conglomerate of amino acids. Mass spectrometers also are advantageous because they provide a very rapid result.

Capillary electrophoresis is a flow-through endpoint detection method that uses an electric field to separate molecules based on size. Molecules in the aliquot being tested are labeled, for example with a fluorescent molecule or radiological label, and flow in the electrical field at a rate that is dependent on the size of the molecule past a detector, for example an optical or radiological detector. Capillary electrophoresis requires only a few minutes to provide a result and requires a small sample volume (usually less than a microliter). Capillary electrophoresis therefore is a preferred detection method for embodiments where frequent sampling from the signal generation reaction are used to provide the most rapid result possible for samples having high concentrations of target.

Since capillary electrophoresis is a microfluidic optical method, it mates well with reactions that have microfluidic formats. Thus, intermittent pumping of the reaction products into the capillary electrophoresis channel can be implemented with relative ease. The reaction products may enter the capillary electrophoresis channel at specifically timed intervals so that later injections do not influence or interfere with the assay of previously injected material. The long delay, which often occurs with amplification and can be up to an hour, during which time no information is reported to the assayer, can be reduced by serially injecting the samples for capillary electrophoresis for rapid assay during amplification.

Protein and nucleic acid microarray detection methods also are contemplated for use with the invention. These methods are useful where identification of a particular species of biological organism is desired because one microarray assay device can test simultaneously for the presence of, for example, several or many specific nucleic acids having different unique sequences. Microarray devices require low volumes for operation, however this method can require longer times to obtain a result unless a mixing strategy such as vibration, thermal excitation/convection, electric field induced changes in the contact angle of the medium (electrowetting), dielectrophoretic manipulation-based mixing or the like is implemented. Although microarrays typically are not quantitative, they usually require only femtomolar concentrations to result in a detectable signal. Thus, intermittent, periodic or continuous injection of reaction products through a microarray chamber can be used to produce a detectable signal prior to the completion of the reaction when the samples contain concentrated amounts of target. Thus, some embodiments of this invention preferably apply to accelerated microarray detection schemes.

Methods for collecting sample aliquots from a signal generation reaction include any known method of collecting an aliquot (small representative fraction) of the fluid of the reaction mixture which are applicable to the small volumes used with the invention. Preferred methods include electrostatic methods (e.g., electrowetting) to split a smaller droplet from a larger one, electrolytic (electrolysis) methods to generate gas bubbles in a confined fluid space which segregate the reaction volume into multiple smaller aliquots and fluid injection methods to introduce an immiscible fluid (e.g., air, perfluorocarbon, or oil) into a sample flow stream to segregate the reaction mixture into multiple small aliquots. Electrolytic methods to generate gas bubbles use two electrodes to produce a gas bubble by electrochemically decomposing water into hydrogen and oxygen gas and are useful in systems using reagents that are insensitive to electrochemical reactions. Alternatively, samples from the reaction chamber can be pumped intermittently, periodically or continuously into a capillary electrophoresis channel without the need for an immiscible interface, since the products from the reaction migrate due to an electric field applied perpendicular to the sample injection channel in these embodiments.

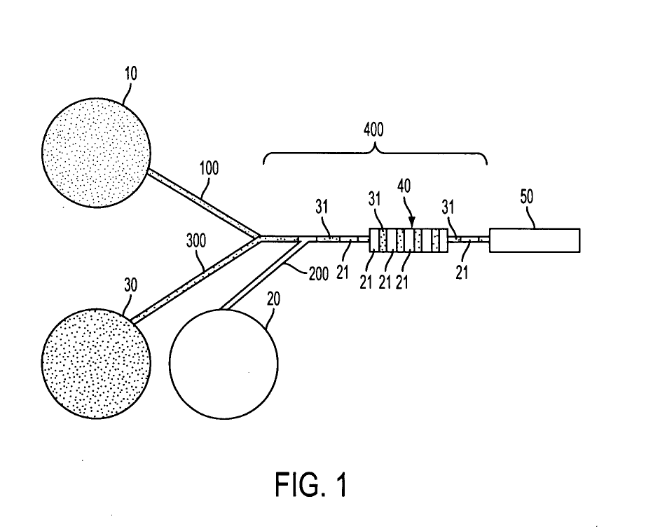

Referring now to the Figures, FIG. 1 is a schematic diagram showing a preferred embodiment of the invention. According to the method and the device pictured in FIG. 1, sample 30 is directed to a flow channel 300. Likewise, reagent mixture 10 is directed to a flow channel 100. Flow channels 100 and 300 conduct their respective contents to a fluid path or conduit 400 where the sample 30 and reagent mixture are introduced into the fluid path or conduit 400 and mixed together to produce a reaction mixture 31. The reagent 10 is any chemical, reagent or mixture of chemicals and reagents which provides the proper environment and starting materials for amplification of target present in sample 30.

For PCR, typical reagents may be 100 mM KCl, 10 mM Tris-HCl (pH 7.4), 0.1 mM EDTA, 1 mM dithiothreitol, 0.5% Tween 20™, 0.5% NP-40, 50% glycerol and oligonucleotides, or any suitable regents known in the art for this purpose. For exponential amplification reactions (EXPAR), a suitable reaction mixture may contain 85 mM KCl, 25 mM Tris-HCl (pH 8.8, 25° C.), 2.0 mM MgSO₄ 5 mM MgCl₂, 10 mM NH₄SO₄, 0.1% Triton X-100, 0.5 mM dithiothreitol, 0.4 U/μl N.BstNBI nicking enzyme, 0.05 U/μl Vent exopolymerase, 400 μM dNTPs, 10 μg/ml BSA, 0.05 μM template and primer oligonucleotides. These reactions generally include target site probes, RNA polymerase, dinucleotide initiator and NTP terminators. SDA reagents may be 1 μM oligonucleotide probe, 6.9 mM tricine (pH 7.6), 50 mM Tris-HCl (pH 8), 10 mM MgCl₂ and 5 mM dithiothreitol, at a temperature of 52.5° C. Those of skill are familiar with these types of reactions and are aware of modifications to such methods, reagents and conditions for these reactions. Such reagent mixtures are well known in the art and may include any buffers, enzymes, nucleic acid building blocks and the like which would be required for target amplification. Any of these reagent mixtures and conditions are contemplated for use with embodiments of the invention.

Referring to FIG. 1, the reagent mixture 10 and the sample 30 each may be contained within vessels (not shown). Reaction mixture 31, once formed, is conducted along a fluid conduit 400 (from left to right as depicted in the exemplary embodiment of the FIGURE) by a means for producing fluid flow (e.g. gravity, a pump, electro-osmosis, capillary action, pressure gradients and the like) or any known means). The terms “fluid conduit” and “fluid path” are essentially interchangeable and indicate any container which can hold, supply or transport the fluid(s) of the method and may be configured as a tube, a vessel of any configuration or may have discrete zones each with different configurations. The fluid path serves to hold fluid while the fluid moves to, into, through, past, out of and/or away from the reaction chamber or which in addition also forms the reaction chamber. In some embodiments, therefore, the reaction chamber and fluid path are integrated such that the reaction chamber is a zone of the fluid path, which optionally is a widened area of the path which forms a vessel of any configuration. The fluid conduit and reaction chamber may be made of any material, flexible or stiff, which does not chemically interfere with the reactions taking place, for example, glass, quartz, metal, plastic and the like.

In alternative embodiments, the sample 30 and reagent mixture 10 are mixed together in a vessel prior to entering the fluid conduit 400, and the flow channels 300 and 100 may be omitted. An immiscible fluid 20 (which is immiscible with the reaction mixture 31) is directed along a fluid conduit 200 to fluid conduit 400 where at intervals, the immiscible fluid 20 is injected into the fluid path through an opening or port in the conduit 400 in small volumes 21 which separate the reaction mixture 31 into aliquots. In alternate embodiments, the immiscible fluid 20 is generated, for example electrolytically, rather than injected or an electrowetting method is used to separate aliquots. Electrolysis or hydrolysis converts a liquid into a gas. For example, when sufficient current flows across two electrodes in an electrolyte, hydrogen and oxygen are produced, resulting in a gas interface. If electrolysis electrodes are placed in or adjacent to microchannels, this method can be used to split a liquid sample into discrete aliquots. Alternatively, a reservoir of an immiscible fluid such as perfluorocarbon or oil can be pumped intermittently into the channel in order to separate samples.

These techniques advantageously work in concert with a pump or other driving force that causes the reagents to flow into the detection channel or chamber. These reaction mixture aliquots 31 and the volumes of immiscible fluid 21 are carried along the fluid conduit 400 and through or into a reaction chamber 40 where the aliquots 31 remain separated from each other. The reaction chamber 40 is disposed in or around the fluid conduit 400 as part of the fluid conduit 400 and provides a zone in the fluid conduit 40 which provides conditions under which amplification or another signal generating reaction can occur. The reaction chamber can be positioned in parallel or serial to the fluidic path that interfaces with the detector 50. Thus, while flowing through the reaction chamber 40, the aliquots 31 are subjected to conditions under which the amplification or other signal generating reaction occurs. The reaction chamber 40 may be, for example, a temperature controlled region or regions, a thermal cycler, isothermal heater or the like. During the residence time of the reaction mixture aliquots 31 in the reaction chamber 40, the amplification reaction takes place, however the flow rate of the aliquots 31 through the reaction chamber 40 optionally is adjusted (e.g., decreased) so that each subsequent aliquot 31 entering the reaction vessel 40 has a longer residence in the reaction vessel 40 than previous aliquots 31.

The flow rate may be periodically halted to provide the desired residence time in the reaction chamber for each aliquot. Thus, there are relatively few amplified products in the first aliquot compared to the final aliquot and the first aliquot is available for assay by the detector quickly. The aliquots 31 then exit the reaction vessel 40 and are continued along the fluid path 400 to a detector 50 where amplified target in the aliquot 31, if present, is detected.

In one embodiment, the reaction chamber is perpendicular to a capillary electrophoresis channel and a sample is periodically injected into the reaction chamber. Alternatively, the sample is split into a number of segments (e.g., 10) and a heater is placed to maintain the desired temperature along the length of the capillary electrophoresis channel. The reaction can proceed, in this embodiment, as it continuously flows and until it reaches the detection zone. Thus, periodic, intermittent or continuous samples are subjected to detection.

In an alternative configuration of this embodiment, the immiscible fluid 20 enters the fluid path 400 to segregate reaction mixture aliquots 31 after the reaction mixture exits the reaction chamber. Therefore, the reaction mixture is subjected to amplification conditions as a single large sample, which then is segregated into individual aliquots 31 at intervals, these aliquots then are conducted to the detector using an adjustable flow so that a portion of the reaction mixture exits the reaction vessel, is collected as an aliquot, and is conducted to the detector after different amplification times. The reaction chamber may be a discrete vessel or a zone in the path and may comprise a thermal cycler, a temperature-controlled region or a series of temperature-controlled regions which can subject a fluid flowing through the regions to cycles of different temperatures as the fluid enters zones of the path which are maintained at these different temperatures.

### EXAMPLE 1

### Biomolecule Detection by EXPAR

An EXPAR reaction proceeds according to the methods of Van Ness et al., Proc. Natl. Acad. Sci. USA 100 (8): 4504-4509, 2003, the disclosures of which are hereby incorporated by reference in their entirety, in a heated chamber. A syringe pump periodically injects an aliquot from the chamber into a capillary electrophoresis detection device. A twin T injection chip, such as are available from Micralyne™ is used to allow periodic or intermittent injections into the capillary electrophoresis detection system. The sample is pumped using a syringe pump at 1 nl/minute for 10 seconds. An electric field is applied perpendicular to the injection channel, which causes the sample to migrate down the channel to the detection zone of the device. Ten samples are injected into the system over a course of 10 minutes.

1. A rapid assay method for detection of a target biomolecule, which comprises:
(a) providing a sample for analysis;
(b) optionally subjecting said sample to analysis for detection of said target biomolecule;
(c) subjecting said sample to biochemical amplification by combining said sample with biochemical amplification reagents to form a biochemical amplification reaction mixture and subjecting said biochemical amplification reaction mixture to conditions wherein biochemical amplification can take place;
(d) simultaneously with (c) collecting at least one sample aliquot of said sample during said biochemical amplification; and
(e) subjecting said sample aliquot to analysis for detection of said target biomolecule.

2. The rapid assay method of claim 1 wherein said target biomolecule is a nucleic acid.

3. The rapid assay method of claim 1 wherein said biochemical amplification is performed using a technique selected from the group consisting of the polymerase chain reaction, strand displacement amplification, the exponential amplification reaction, and abscription.

4. The rapid assay method of claim 1 wherein said analysis for detection is selected from the group consisting of capillary electrophoresis and mass spectrometry.

5. The rapid assay method of claim 1 wherein said sample aliquot collection comprises subjecting said biochemical amplification reaction mixture to fluid transport along a fluid conduit and separating discrete volumes of said biochemical amplification reaction mixture from each other to form aliquots by introducing an immiscible fluid at intervals in said fluid conduit.

6. The rapid assay method of claim 5 wherein said sample aliquot collection occurs prior to said biochemical amplification.

7. The rapid assay method of claim 5 wherein said sample aliquot collection occurs after said biochemical amplification begins.

8. The rapid assay method of claim 1 wherein said sample aliquot collection is episodic.

9. The rapid assay method of claim 1 wherein said sample aliquot collection is periodic.

10. The rapid assay method of claim 1 wherein said sample aliquot collection is continuous.

11. An assay device for rapid assay of a sample for presence of a biomolecule target which comprises:
(a) a hollow fluid conduit comprising a first open end, a second open end and an opening in said conduit between said first end and said second end to admit a fluid into said fluid conduit;
(b) a means for producing fluid flow in said fluid conduit in the direction from said first end to said second end;
(c) a means for introducing an amplification reagent mixture into the first end of said fluid conduit and a means for introducing said sample into the first end of said fluid conduit to mix said amplification reagent mixture and said sample together to form a reaction mixture in said fluid conduit;
(d) a reaction chamber disposed in said fluid conduit between said first end and said second end, wherein said reaction chamber provides conditions under which amplification of said biomolecule target can occur;
(e) an aliquot collection means that introduces a fluid into said fluid conduit at intervals, wherein said fluid is immiscible with said reaction mixture and wherein said fluid separates said reaction mixture into discrete aliquots of reaction mixture; and
(f) a detector, detachably connected in a fluid conducting manner to the second end of said fluid conduit.

12. The assay device of claim 8 wherein said aliquot collection means is selected from the group consisting of a fluid injector, an electrostatic droplet splitter and an electrolytic gas generator.

13. The assay device of claim 8 wherein said detector is selected from the group consisting of a mass spectrometer, a capillary electrophoresis device with an optical detector and a microarray.

14. An assay device for rapid assay of a sample for presence of a biomolecule target which comprises:
(a) a hollow fluid conduit comprising a first open end, a second open end and an opening in said conduit between said first end and said second end to admit a fluid into said fluid conduit;
(b) a supply of amplification reagent to the first end of said fluid conduit;
(c) a supply of said sample to the first end of said fluid conduit;
(d) a mixer to mix said amplification reagent and said sample;
(e) a pump;
(f) a reaction chamber disposed in said fluid conduit between said first end and said second end, wherein said reaction chamber provides conditions under which amplification of said biomolecule target can occur;
(e) an aliquot collector; and
(f) a detector, detachably connected in a fluid conducting manner to the second end of said fluid conduit.
